(19) United States
(12) Plant Patent Application Publication
(10) Pub. No.: US 2002/0092077 P1
(43) Pub. Date: Jul. 11, 2002

(54) Chrysanthemum named CHEOPS

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/734,599

(22) Filed: Dec. 13, 2000

(51) Int. Cl. A01H 5/00

(52) U.S. Cl. PLT/297

(57) ABSTRACT

A chrysanthemum plant named CHEOPS characterized by its medium sized blooms with soft pink ray florets and prolific branching; natural season flower date August 21-27; blooming for a period of 4 weeks.

### BACKGROUND OF THE INVENTION

The new plant of the present invention comprises a new and distinct cultivar of Chrysanthemum plant that is a cross of unknown Dendranthema Grandiflora parents. The new and distinct cultivar was a seedling resulting from the open pollination among groups of chrysanthemum cultivars maintained under the control of the inventor for breeding purposes. The new and distinct cultivar was discovered and selected as one flowering plant by Rob Noodelijk on a cultivated field in Rijsenhout Holland in September 1997. The plant has been asexually reproduced by cuttings in greenhouses at Rijsenhout Holland. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

FIG. 2 shows the various stages of bloom and foliage of the new cultivar.

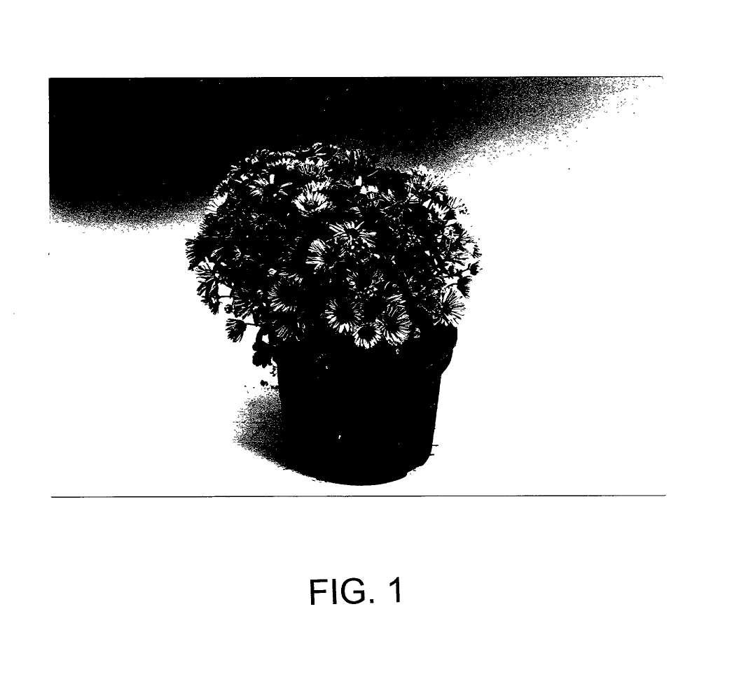

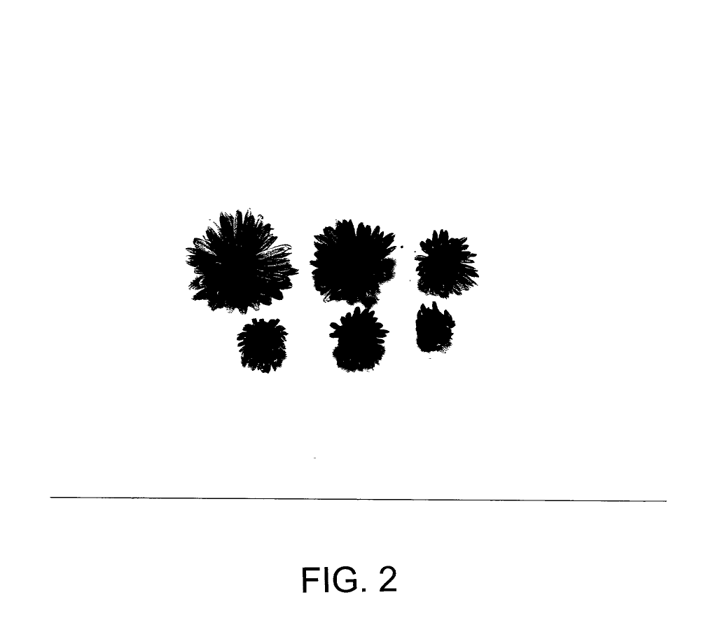

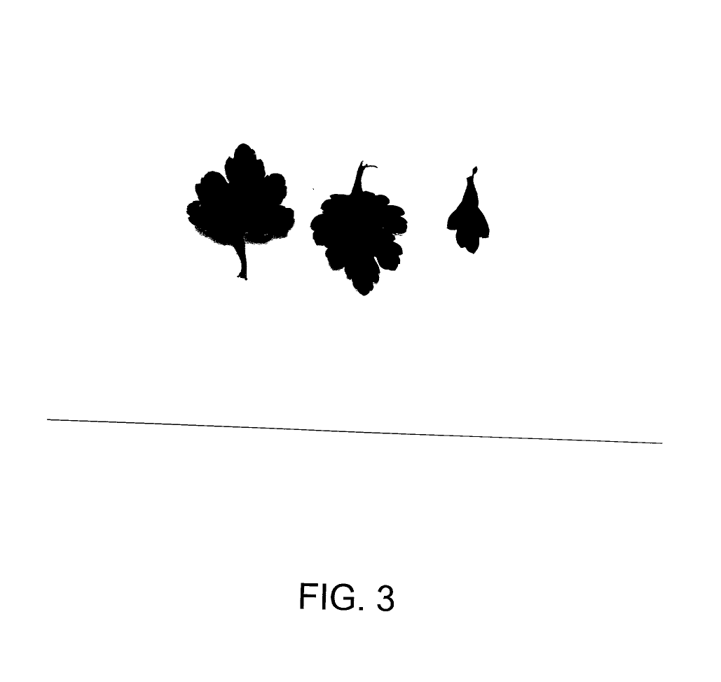

### DESCRIPTION OF THE INVENTION

This new variety of chrysanthemum is of the botanical classification Dendranthema Grandiflora. The observations and measurements were gathered from plants grown out door in Rijsenhout, Holland under natural day length and temperature and planted week 22 in 1998 and 1999. The natural blooming date of this crop was August 21-27 (week 34). The average height of the plants was 30-35 cms. This new variety produces medium sized blooms with soft pink ray florets and yellow disc-florets blooming for a period of 4 weeks.

The following is a description of the plant and characteristics that distinguish CHEOPS as a new and distinct variety.

The color designations are taken from the plant itself. Accordingly, any discrepancies between the color designations and the colors depicted in the photographs are due to photographic tolerances.

Botanical classification: Cultivar cheops

Bud.—

Size.—medium, cross-section +/−1.2 cm height +/−1.0 cm.

Outside color.—Purple 75B.

Bloom.—

Size .—Medium.

Fully expanded.—5.5-6.0 cms.

Borne (number of blooms per branch).—Upper and lower portion plural blooms per branch (approx. 4-5 blooms per branch).

Length of lateral branch.—From top till bottom 10-12 cm.

Lateral branch, attachment.—Strong.

branching.—Prolific with 5-6 breaks after pinching.

Form.—Daisy.

Performance (on the plant).—4 weeks.

Color.—

Center of flower.—Immature yellow-green 153C Mature yellow-orange 16A.

Color of the upper surface of the majority of the ray-florets.—Red-purple 74D.

Color of the lower surface of the majority of the ray-florets.—Purple 75B.

Tonality from distance.—A daisy garden mum with soft pink blooms.

Discoloration.—To purple 75D.

Ray florets.—

Texture.—Upper side and under side smooth.

Number.—36-40.

Cross-section.—Flat.

Longitudinal axis of the majority.—Straight.

Shape of tip.—Rounded sometimes dentate.

Fragrance.—Typical chrysanthemum.

Disc diameter (natural season crop when visible).—1.2 -1.4 cm.

Reproductive organs.—

Stamen.—Yellow, thick, +/−4 mm in length.

Pollen.—Present.

Styles (present in both ray and disc florets).—Green, thin.

Style length.—+/−4 mm.

Stigmas.—Yellow-green.

Stigma width.—+/−1 mm.

Ovaries.—Enclosed in calyx.

Plant.—

Form.—A garden mum (pot mum) meant for out door use.

Growth.—Medium.

Height (from soil level).—30-35 cms.

Stem color.—Green 138A.

Stem strength.—Strong.

Stem brittleness.—Present.

Stem anthocyanin coloration.—Present.

Natural blooming date.—August 21- 27.

Foliage.—

Color.—Upper side green 137A Under side green 137C.

Size.—Very small, length +/−4.5 cm width +/−4.0 cm.

Quantity (number per lateral branch).—8-10.

Shape.—Egg shaped.

Texture.—Fleshy.

Ribs and veins.—Ribs and veins not so well developed.

Edge.—Lobed.

Shape of base of sinus between lateral lobes.—Acute.

Margin of sinus between lateral lobes.—Converging.

Shape of base.—Truncate sometimes asymmetrical.

Apex.—Cuspidate.

I claim:

1. A new and distinc variety of chrysanthemum plant as described and illustrated
